(19) United States
(12) Patent Application Publication
(10) Pub. No.: US 2025/0256158 A1
(43) Pub. Date: Aug. 14, 2025

(54) Artificial Intelligence Apparatus and Method for monitoring prescribed Movements

(71) Applicants: Hoda Salsabili, West Lafayette (IN); MOHAMMAD HOSSEINI, West Lafayette, IN (US)

(72) Inventors: Hoda Salsabili, West Lafayette (IN); MOHAMMAD HOSSEINI, West Lafayette, IN (US)

(21) Appl. No.: 18/439,044

(22) Filed: Feb. 12, 2024

(51) Int. Cl. A63B 24/00; A63B 71/06; G06Q 50/00; G06V 40/10; G06V 40/20; G16H 10/20; G16H 20/30; G16H 50/20

(52) U.S. Cl. CPC A63B 24/0062; A63B 24/0006; A63B 24/0075; A63B 71/0622; G06Q 50/01; G06V 40/10; G06V 40/23; G16H 10/20; G16H 20/30; G16H 50/20; A63B 2024/0009; A63B 2220/806; A63B 2230/00

(57) ABSTRACT

This invention provides an artificial intelligence (AI) apparatus and method for prescription, real-time monitoring and tracking individualized physical therapy exercise plan and classes to remote users. In one aspect, the present disclosure comprising a method utilizing the AI apparatus for prescribing body movements to individuals and/or groups that can be accessed via a digital communication network by a user at a remote location, sending digital video and audio content comprising AI-prescribed individualized exercises and classes from a server to a personal digital device or a computer associated with the system, AI-powered tracking and sending real-time feedback for the correct pattern of body movements, providing a social platform for communication among users or users-physical therapist, and a summarized AI-generated progress note of the user.

### CROSS-REFERENCE TO RELATED APPLICATION

This Patent Application is continuation-in-part patent to U.S. patent application Ser. No. 18/683,058 filed on Feb. 12, 2024 and PCT/US22/40130 filed on Aug. 11, 2022 which claims priority to Provisional Application: 62/232357 filed Aug. 12, 2021, the disclosure of which is considered part of the disclosure of this application and is hereby incorporated by reference in its entirety.

### FIELD OF THE INVENTION

This invention provides a systems, methods, and apparatus for monitoring and tracking individualized physical therapy exercise plan of remote users. In one aspect, the invention relates to an artificial intelligence (AI) apparatus for prescribing body movements to individuals and a monitoring system with real-time feedback and social communication among users.

### BACKGROUND

In the physical therapy industry, in home therapeutic exercises are commonly assigned to patients and individuals to help ensure and improve physical recovery when in-person therapy session is not available or used. Typically, adherence of patients to plan of care and exercises is very low, approximately 35%. Low adherence indicates the patients do not complete the recommended therapy sessions or do not perform exercises regularly at home or in the clinic. For physical therapist (“PT”), low adherence lowers profit for missing sessions and increases denied claims for lack of progress in the patients' recovery. For insurance companies, low adherence costs higher rate of rehospitalization and disability.

Some factors concluding to low adherence of patients are poor tracking of mental and physical pain and performance during and after exercises, lack of feedback or correct instruction, little motivation and social engagement, and lack of planned and various exercises for prescribed exercises. Often physical therapists or trainers print exercises on a paper, give verbal or video prescription of exercise or in the best scenario, they use some commercial cell-phone applications which has extensive limitations to monitor patient/trainee specifically out of clinic. These methods leave exercises for patients without any real-time biomechanical feedback analysis for correct performance, biofeedback for physiological progress or any objective report that summarize the progression of patient/trainee by exercises. Additionally, the limitation in the number and category of prescribed exercises does not allow the physical therapist to make a comprehensive exercise plan for the patient/trainee. An appropriate communication between physical therapist/trainer with the patient/trainee is also missing. For example, using tele-communication between patient and physical therapist and rating pain and performance in comparison with the group of people with similar complications for the prescribed exercises or attending an exercise class can essentially help tracking of pain and performance for patients. Moreover, the therapy environment is not often very interactive and social engagement with other patients is very limited that reduces the motivation of patients to adhere to their plan of care. An environment that a group of patients practice together with a social interaction capacity would solve the motivation needed for a better adherence.

An artificial intelligence apparatus for prescription and monitoring the exercise at home or remote location that patients objectively see their progress, engage in social environment and become aware of their body performance during exercises can subsequently increase the adherence of patients to their plan of care and benefit them for a better recovery. It also let the physical therapist to follow the patient progress using artificial intelligence, increase the patients' recovery, and encourage the patients to attend all prescribed sessions in the plan of care effectively and objectively. Additionally, the AI apparatus of the present disclosure can prescribe exercises and monitoring to help insurance companies to reduce the cost of non-adherence including higher rate of rehospitalization and disability in non-adhered patients to their plan of care and request for an objective progress note from the physical therapists.

There exists a need for an AI apparatus system and method that allows physical therapist/trainer to monitor and engage the patients/clients in a private and/or class and social environment to motivate the exercise performance at remote locations. Furthermore, the AI apparatus of the present disclosure can reduce the amount of hours with a physical therapist or potentially eliminate the need for a physical therapist all together.

### BRIEF SUMMARY OF THE INVENTION

In one aspect, the present disclosure related to an AI apparatus and method for prescribing guided exercise routine to a user by AI-driven movement monitoring system. The AI apparatus of the present disclosure can prescribe movement according to the predefined initial evaluations comprising questionnaires, AI-driven motion analysis assignments and evaluations. The predefined initial evaluation and diagnosis can be conducted by a pre-determined physical therapist. AI-prescribed movements can be provided to a remote user and can be accessed via a digital communication network by the pre-determined user to which the pre-determined exercise routine corresponds. An interface can be provided that can provide a display to the user and a camera. The display can allow a pre-determined user to access the pre-determined AI-driven initial evaluation exercises and is the camera can be configured to record and/or monitor one or more movements of the pre-determined user while participating in the pre-determined exercise routine. Audio and/or audiovisual data can be received and/or recorded of the one or more movements of the pre-determined user. The one or more initial evaluation movements can then be analyzed with respect to a movement database and/or through artificial intelligence/machine learning modules for tracking patients performance and/or through one or more pre-determined movement parameters assigned to the pre-determined user. Upon completion of the pre-determined initial evaluation, a post-performance AI-guided written feedback report can be provided to the pre-determined user and also the AI-movement monitoring system prescribes one or more exercises, wherein the AI-driven movement monitoring system connected to the exercise database, initial evaluation database, and post exercise performance progress notes. The AI-movement monitoring system get updated for initial evaluation database.

In another aspect the present disclosure relates to a method for monitoring the AI-prescribed movements to patient/users(s). An interface can be provided that can a display and a camera, wherein the display can allow the pre-determined user to access the pre-determined AI-driven exercise routine and is configured camera for recording and/or monitoring one or more movements of the pre-determined user while participating in the pre-determined exercise routine. Audio and/or audiovisual data can be received and/or recorded of the one or more movements of the pre-determined user. The one or more movements can then be analyzed with respect to an exercise movement database and/or through artificial intelligence/machine learning methods for tracking patients performance and/or through one or more pre-determined movement parameters assigned to the pre-determined user. An assessment, score, and/or rating can be assigned to each of the recorded movements and based upon the assessment and/or analysis real-time feedback can be transmitted to the pre-determined user. Upon completion of the pre-determined exercise routine post-performance feedback can be provided to the pre-determined user as a progress note. Additionally, the post-performance feedback can be transmitted and/or communicated to a AI-movement monitoring system. The AI apparatus of the present disclosure can generate a performance report and transmit the report to the user and/or the instructor upon completion of the prescribed exercise(s) based upon the performance of the user. The AI-driven apparatus can update the post performance progress note, and the movement exercise database according to the patient/user real time and generated feedback.

In another aspect the present disclosure relates to a method for providing group physical therapy classes from a server to one or more users through a personal digital device or to a computer associated with the system. The method and system can track and send real-time feedback for the correct pattern of body movements using artificial intelligence/machine learning via the system or a Tele-Physical therapist (PT). The classes/instruction can similarly provide a social platform for exercising group of patients with similar complication and under the supervision of a Tele-PT.

In yet another aspect, the present disclosure relates to an artificial intelligence apparatus for prescribing and evaluating exercise routings to one or more users. The apparatus can include a processor and a memory. The memory can be configured to store one or more inputs and user data and other databases. Similarly, the memory can be communicatively coupled to a network to access additional information and memory modules. The processor can similarly be communicatively coupled to a network. The processor can be configured to provide access to a remote user to complete an initial eval evaluation comprising a general health and performance questionnaires and initial diagnosis. The process can then provide access to the user to perform pre-determined AI-guided assessments to be recorded by a camera and one or more physiological sensors are communicatively coupled to allows user to access the assessments, to perform AI-driven assessment of joints range of motion, muscular strength and performance. An initial evaluation response output comprising a recorded video and physiological data from the one or more sensors can be received from the user device. The initial evaluation response output can be analyzed with respect to a movement database, initial evaluation database to extract one or more pre-determined movement parameters. One or more exercise routines can be prescribed to the user, wherein the AI prescription is established with respect to one or more of the following: movement database, movement assessments, other user progress notes, exercise database, and initial evaluation database. Information can then be provided to the user via display about one or more prescribed exercise routines of the AI prescription that can be accessed via the network.

The AI prescription can be transmitted to a user interface which can include a display and a camera, wherein the display allows user to access the AI-prescribed exercise routine and the camera is configured to capture video data of the one or more movements of the user and the physiological sensor is configurated to collect performance data while participating in the exercise routine by the user camera. One or more movements preformed by the user can be analyzed with respect to initial evaluation databases and one or more pre-determined movement parameters. The performance of the AI-prescription can be determined with respect to the initial evaluation databases and movement parameters through the AI module and movement analysis engine. The apparatus can then generated and provide real-time feedback to the user based upon the performance of the prescribed movement. The real-time feedback can include one or more of automated feedback response from the AI module, real-time feedback using the AI module comparing a measured movement parameter to a target movement parameter, and feedback based upon the movement of the user and analysis of the user movement or a group of users. The feedback provided to the user can be visual, auditory, and/or a haptic modality.

In yet another aspect, the present disclosure relates to a real-time movement tracking system for monitoring prescribed movements assigned to a user to provide real-time feedback during the performance of the prescribed movement in a remote location.

In yet another aspect, the present disclosure relates to a method for providing a live and/or archived exercise routine group courses to and a platform for social communication among remote users by first providing information to one or more remote users about one or more pre-determined AI-prescribed exercise routines established by a AI-driven movement monitoring system, and second, by providing social interaction among users that can be accessed via a digital communication network by one or more remote users.

### BRIEF DESCRIPTION OF THE DRAWINGS

FIG. 1A is a block diagram of an exemplary system of the present disclosure.

FIG. 1B is a block diagram of an exemplary system of the present disclosure.

FIG. 2A is a diagram of an exemplary embodiment of the method of the present disclosure.

FIG. 2B is a diagram of an exemplary embodiment of the method of the present disclosure.

FIG. 3 is an illustration of a user interface for a patient utilizing an exemplary embodiment of the present disclosure and social interaction with other users.

FIG. 4 is an illustration of a user interface for a patient utilizing an exemplary embodiment of the present disclosure for viewing schedule classes.

FIG. 5 is an illustration of a user interface for a patient utilizing an exemplary embodiment of the present disclosure showing a user's prescribed individualized classes and available group classes

FIG. 6 is an illustration of a user interface for a patient utilizing an exemplary embodiment of the present disclosure during an individualized prescribed class and performance of a prescribed movement that analyzed with artificial intelligence/machine learning.

FIG. 7 is an illustration of a user interface for a patient utilizing an exemplary embodiment of the present disclosure for the group class and social platform.

FIG. 8 is an illustration of a user interface for a patient utilizing an exemplary embodiment of the present disclosure showing progress illustrations for various prescribed exercises.

### DETAILED DESCRIPTION OF THE INVENTION

The following detailed description includes references to the accompanying drawings, which forms a part of the detailed description. The drawings show, by way of illustration, specific embodiments in which the invention may be practiced. These embodiments, which are also referred to herein as “examples,” are described in enough detail to enable those skilled in the art to practice the invention. The embodiments may be combined, other embodiments may be utilized, or structural, and logical changes may be made without departing from the scope of the present invention. The following detailed description is, therefore, not to be taken in a limiting sense.

Before the present invention of this disclosure is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the disclosure made herein.

Unless otherwise indicated, the words and phrases presented in this document have their ordinary meanings to one of skill in the art. Such ordinary meanings can be obtained by reference to their use in the art and by reference to general and scientific dictionaries.

References in the specification to “one embodiment” indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The present disclosure can provide one or more embodiments that may be, among other things a method, system, or computer program and can therefore take the form of a hardware embodiment, software embodiment, or an embodiment combining software and hardware. In one exemplary embodiment, the present invention can include a computer-program product that can include computer-useable instruction embodied on one or more computer readable media.

Computer-readable media include both volatile and nonvolatile media, removable and nonremovable media, and contemplates media readable by a database, a switch, and various other network devices. Network switches, routers, and related components are conventional in nature, as are means of communicating with the same. By way of example, and not limitation, computer-readable media comprise computer-storage media and communications media.

Computer-storage media, or machine-readable media, include media implemented in any method or technology for storing information. Examples of stored information include computer-useable instructions, data structures, program modules, and other data representations. Computer-storage media include, but are not limited to RAM, ROM, EEPROM, flash memory or other memory technology, CD-ROM, digital versatile discs (DVD), holographic mediator other optical disc storage, magnetic cassettes, magnetic tape, magnetic disk storage, and other magnetic storage devices. These memory components can store data momentarily, temporarily, or permanently.

The various components can include a communication interface. The communication interface may be an interface that can allow a component to be directly connected or communicatively connected to any other component or allows the component to be connected to another component over network. Network can include, for example, a local area network (LAN), a wide area network (WAN), cable system, telco system, or the Internet. In an embodiment, a component can be connected to another device via a wireless communication interface through the network, including but not limited to cellular mobile communication (such as 2G, 3G, 4G and 5G networks), Bluetooth Low Energy, WiFi, Near Field Communication (NFC), Ultra Wideband (UWB), or other radiofrequency means. Some exemplary embodiments of the assessments and tracking system of the present disclosure and method may be described in the general context of a computer-executable instruction, such as program modules, being executed by a computer. Generally, program modules may include routines, programs, objects, components, data structures, among other modules, that may perform particular tasks or implement particular abstract data types. The various tasks executed in the system may be practiced by distributed computing environments where tasks are performed by remote processing devices that are linked through communications network, which may include both local and remote computer storage media including memory storage devices and data.

Artificial intelligence refers to the field of studying artificial intelligence or methodology for making artificial intelligence, and machine learning refers to the field of defining various issues dealt with in the field of artificial intelligence and studying methodology for solving the various issues. Machine learning can be defined as an algorithm that enhances the performance of a certain task through a steady experience with the certain task.

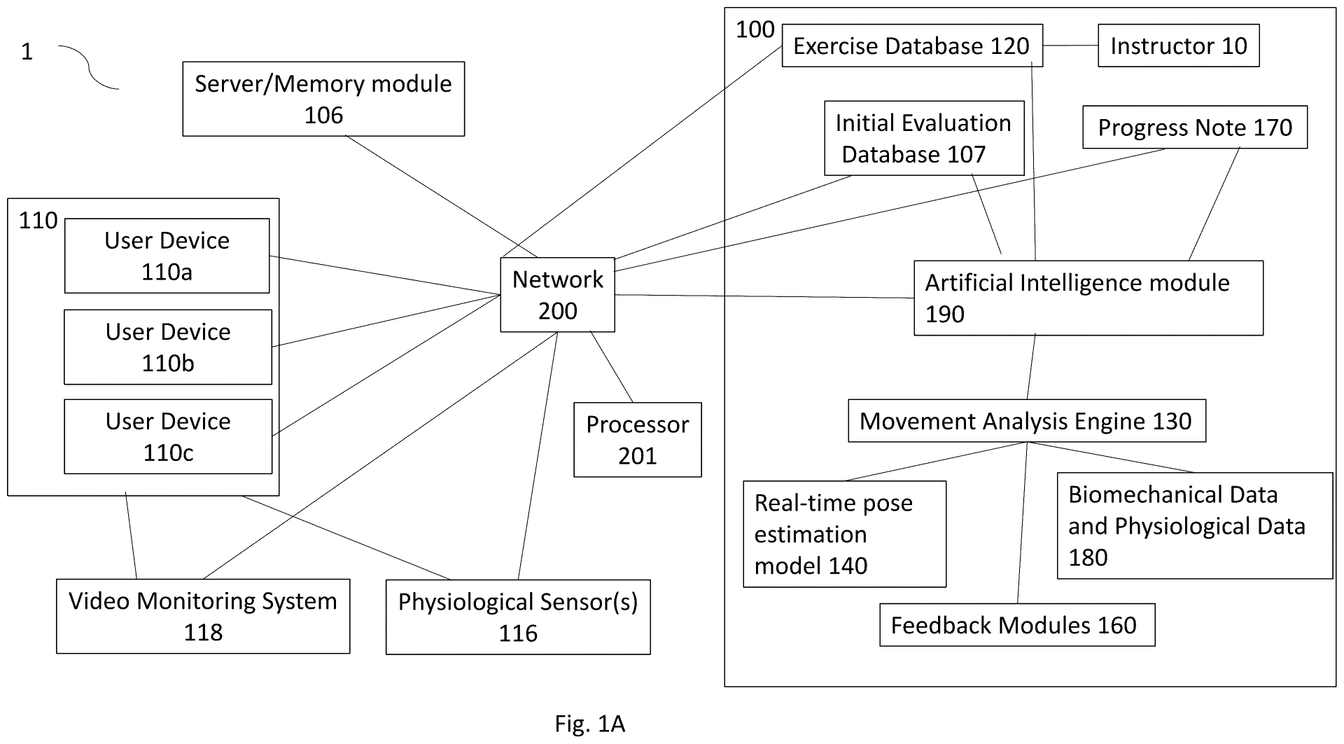

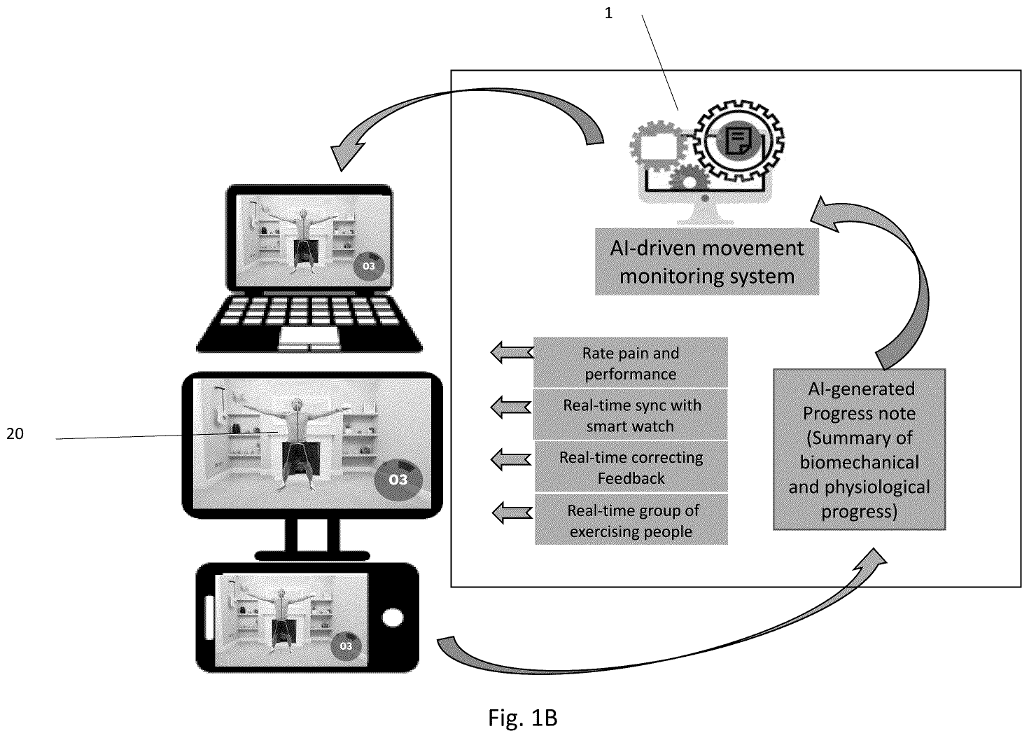

As shown in FIGS. 1A-B, in one exemplary embodiment of the present disclosure, a AI-driven movement monitoring apparatus 1 of the present disclosure can include one or more software/applications including an exercise database modules 120 for prescribing exercises to one or more users. The artificial intelligence module 190 can access one or more of the various databases including the exercise database 120. The exercise database 120 can include a variety of categories of exercises for different groups of users and selects appropriate measures to report the progress in each exercise to the physical therapist/instructor. Each exercise can additionally include one or more movement parameters. A user specific goal measurement can be assigned to the one or more movement parameters for each of the related exercises assigned to the user. The various exercises, exercise programs, and corresponding movement parameters can be stored on a memory or server and customized into one or more pre-determined training modules/programs to be assigned by an AI module 190 for a user 20.

The system 1 can communicate with a manufactured hardware/or patient personal user device 110 such as cellphone, tablet or laptop. An integrated graphical display or separate graphical display can be communicatively coupled to the user device 110 can display the exercises and a video monitoring system 118 can collect video content including real-time biomechanical and 116 physiological data as a user performs an exercise. The method and system of the present disclosure can monitor AI-prescribed movements through a movement analysis engine 130 that can include a human pose estimation model 140 and real-time movement feedback module using image analysis by artificial intelligence/machine learning module 160 which comparing with the biomechanical and physiological data 180 received via user interface. The apparatus 1 can then provide real-time auditory, visual, and vibrating tactile feedback during exercises to correct pattern of exercise performance.

The apparatus 1 can utilize any computing means such as cloud computing, server, 106, or computing through a user device 110, or separate processing means 201. The computing means may include an address/data bus that is configured to communicate information. Additionally, one or more data processing units, such as a processor (or processors), are coupled with the address/data bus. The processor can be configured to process information and instructions. In one exemplary embodiment, the processor can be a microprocessor. Alternatively, the processor may be a different type of processor such as a parallel processor, application-specific integrated circuit (ASIC), programmable logic array (PLA), complex programmable logic device (CPLD), or a field programmable gate array (FPGA).

The memory can also include data storage units, such as volatile memory unit (e.g., random access memory (“RAM”), static RAM, dynamic RAM, etc.) coupled with the address/data bus, wherein a volatile memory unit is configured to store information and instructions for the processor. The computer system further may include a non-volatile memory unit (e.g., read-only memory (“ROM”), programmable ROM (“PROM”), erasable programmable ROM (“EPROM”), electrically erasable programmable ROM “EEPROM”), flash memory, etc.) coupled with the address/data bus, wherein the non-volatile memory can be configured to store static information and instructions for the processing means. In other exemplary embodiments, the computer system may execute instructions retrieved from an online data storage unit/memory such as in “Cloud” computing or virtual service. In some exemplary embodiments, the computer system can include one or more interfaces, such as an interface, coupled with the address/data bus. The one or more interfaces are configured to enable the computer system to interface with other electronic devices and computer systems. The communication interfaces implemented by the one or more interfaces may include wireline (e.g., serial cables, modems, network adaptors, etc.) and/or wireless (e.g., wireless modems, wireless network adaptors, etc.) communication technology.

The computing system can further be communicatively coupled to one or more other devices such as a tablet, smart phone, camera, personal computers, laptops among others. In some exemplary embodiments, the system can provide information to a user or instructor via a graphical display that may be incorporated into a user device 110 or communicatively coupled to the device 110. The graphical display can be any suitable means including but not limited to a mobile phone, tablet, glasses, smart watch, projector, television, video monitor or any other device capable of displaying video content. In some exemplary embodiments, the user device can include a processing means, a transceiver for communications to a network, and a graphical display for the user. In other exemplary embodiments, a server can function as a processing means for analysis of any audiovisual data captured during the performance of a movement by a user.

The processing means and computer of the present disclosure is one exemplary embodiment in accordance with the system of the present disclosure and is not meant to be a limiting example a computer system. The present disclosure provides a computer/processing system represents a type of data processing analysis that may be used in accordance with various aspects described herein. The processing means and/or computers system can be used to implement computer-executable instructions, such as program modules, being executed by a computer by one or more processors. In some exemplary embodiments program modules include routines, programs, objects, components and/or data structures that are configured to perform particular tasks or implement particular abstract data types. The system of the present disclosure can be utilized to distribute through one or more computing environments, where tasks can be performed by remote processing devices that are linked through a communications network, or such as where various program modules are located in both local and remote computer-storage media including memory-storage devices, virtual databases, or memory modules/servers. Similarly, the system can record and process tasks being executed via one or more devices having a camera to collect audio and/or video data. The databases can include a library of exercises and/or movements that can be used to support a variety of categories of training needed for different groups of patients/trainees that any intended exercises by the physical therapist/trainer can be prescribed.

As shown in FIGS. 1A-B, the system can include one or more computing devices. The computing devices can include a processor means 201 and/or a memory 106. In some exemplary embodiments, the computing device can be communicatively coupled to one or more networks 200. In some exemplary embodiments, a computing device can be an external server 106 or a user device 110, or separate processor 201. The apparatus of the present disclosure can additional be connected to one or more servers/cloud storage databases via the network 200. Additionally, the system can designate, assign, and or store one or more users which can include patients. Each of the patient/users can have a pre-determined device code and account number for communicating with the apparatus 1. The apparatus 1 can further include a video monitoring system 110 such as a camera, video camera, smart phone, or other device that can capture images and/or videos of one or more users performing various AI-driven exercises or prescribed movements from the AI-driven movement monitoring apparatus. In some exemplary embodiments, the video monitoring system 118 can be part of or incorporated into a user device 110, including but not limited to a smart phone, computer, or tablet.

As shown in FIGS. 1A-B, in some exemplary embodiments, the apparatus 1 can utilize and be communicatively coupled to various application databases 100, including but not limited to a movement analysis engine 130 that can include one or more feedback modules 160, artificial intelligence/Machine learning modules 190, or other programs, models, or algorithms for assessing and tracking movement of a user 20 to make an assessment of their performance of the AI-guided initial evaluation and AI-prescribed rehabilitation program or prescribed exercises. The application databases 100 can be communicatively coupled to the a processor via the network 200. The apparatus 1 can provide real-time feedback to the remote user as the user is performing the prescribed or selected exercises. The feedback modules 160 and/or programs can include rate pain and performance module, a real-time sync to timer program, a real-time correcting feedback module, and a group of users exercising with a real-time analysis of individual exercise. In some exemplary embodiments, the apparatus 1 can obtain biomechanical data and/or physiological data 180 and process the data by utilizing a human pose estimation model 140 of the user movement video content transmitted from the video monitoring system 118. Additionally, one or more analysis programs included but not limited to deep learning and artificial intelligence methods 190 can further generate feedback to be transmitted to the user in real-time. The system and method can be improved and automated through various techniques including computer vision, artificial intelligence, deep learning, and/or sensor (IMU) data in addition to text data: questions asked by a human agent, or an artificial intelligence bot-based on sent images, videos and previous answers as well as answers by the consumer from a smartphone and/or other device.

The AI apparatus of the present disclosure can utilize data obtained to assess and analyze the performance of the remote users without an evaluator physically present. In some exemplary embodiments, the AI apparatus 1 of the present disclosure can provide biomechanical and physiological data back to an instructor/therapy professional and/or the user for further analysis and for real-time feedback, or for documenting and approving completion of a prescribed program. Additionally, the system can provide a score/rating to an evaluator and/or the user. The rating can be stored on a memory 106 for further comparison in subsequent sessions/programs performed by the user. The score or rating can include any suitable rating system (i.e. scale of 1-10, 1-100, 5 star rating, etc.). In some exemplary embodiments, a rating can be generated for each of the prescribed movements individually and/or for the entire routine/session of movements. The rating and/or analysis generated by the system can be utilized to determine whether or not and/or what type of real-time feedback to provide to a user. The rating can similarly be utilized to compare an individual user with other users having similar injuries or that have participated in similar movements/exercises. The rating can be based on one or more metrics including number of repetitions completed, number of repetitions completely correctly, assessment of measured movement parameter versus the goal movement parameter, etc.

Real-time feedback can be generated and provided to a user and/or group of users in the class using artificial intelligence and/or machine learning during the performance of the one or more prescribed movements. This feedback can help correct and/or ensure that the user is properly performing the prescribed exercise. In some embodiments, the real-time feedback can be generated and determined based upon measured movements of a user as they are performing prescribed movements against a predetermined goal movement parameter for the prescribed user. At the end of each specific exercise and/or at the end of the entire routine prescribed, a user can additionally provide a pain rating that can be communicated and/or stored for review by a third party. Similarly, the pain rating can also be included in the final post-performance feedback report generated by the system 1.

An instructor 10 can create an exercise database or rehabilitation program/module in the exercise database 120 that can then be assigned or prescribed by AI-driven monitoring system and module 190 to a user 20. The user 20 can access the program through a device communicatively coupled to a network 200 by utilizing the pre-determined login information assigned to the individual user 20. In some exemplary embodiments, the device 110 can be a personal computer such as a tablet, laptop, phone, or other device. The system can then initiate the AI-prescribed rehabilitation program corresponding to the applicable user. In some exemplary embodiments, the system can further include one or more to display instructions to the user and or obtain additional data, such as physiological data. In some exemplary embodiments, the secondary device can be a smart watch or additional sensor devices or monitoring straps. The physiological data can include but not limited to heart rate, blood oxygen level, step count, among others. The video monitoring system 118 can then monitor and track the movement of the user as the user performs the one or more prescribed movements. The system can utilize the video content/data obtained from the video monitoring system to generated real-time feedback to the user as they are performing the movements. In some exemplary embodiments, the system can utilize a pose estimation module to compare the obtained data to the pose model and can additionally be compared with the prescribed parameters set forth for the particular exercise for the specific user. The artificial intelligence module 190 and human pose estimation module 140 can utilize any suitable methods including human pose estimation, API processing, and blaze pose modeling among others. The video monitoring system 118 can obtain real-time biomechanical data during the exercises and can synchronize with the physiological device to collect the real-time physiological data in tandem with the video data. The various data and analysis of the movements by the system can be utilized to provide the real-time feedback to the user. Additionally, in some exemplary embodiments, the data and movement analysis can be utilized to provide a rating to the user for the prescribed movement and/or for the performance of the entire routine.

In one exemplary embodiment, a user/patient can first meet with an instructor/therapist for an initial assessment to get a diagnosis and established initial evaluation that later can be inserted to the AI apparatus 1 of the present disclosure. The apparatus 1 can then later prescribe movements and exercises accordingly. Each of the one or more movements can have an initial measured parameter associated with the movement, which can be used as an initial movement parameter(s) for the prescribed movement. Additionally, a goal movement parameter can be assigned to the movement for the prescribed user 20. In some embodiments, as a user progresses, the apparatus 1 can revise or reassess the user's goal movement parameter. As the user performs the movements remotely, a video monitoring device 118 that is communicatively coupled to a the user device 110 can obtain and analyze the user performing the prescribed movements.

In one exemplary embodiment, a user/patient can first input answers into an assessment questionnaire that can be conducted through the steps of initial assessment according to the initial evaluation database 107 by the apparatus 1. Once a user initiates response inputs into the questionnaire and locate the body site of assessment, the apparatus 1 can drive the patient to the related body site initial assessments according to the initial evaluation database 107. The initial assessment can be completed by a user through using the user device for the assessments included but not limited to joints range of motion, muscle strength assessment against gravity, visual images of the tender location, standing balance and performance, and walking balance and performance assessments. The AI module 190 can compare the collected biomechanical and physiological data 180 via user devices 110 during the initial assessments to the initial evaluation database 107 using movement analysis engine 130 to determine the prescription of exercises. The AI module 190 can then prescribe exercise routine and rehabilitation program to user/patient according to the exercise database 120.

Additionally, in some exemplary embodiments, the system can provide real-time tele-communication with a virtually located instructor 10, which can include but is not limited to a movement evaluator, trainer, physical therapist, occupational therapist, or any other third party for supervision and feedback as well based upon the movement execution by the patient/user 20. An instructor 10 can be assigned or select a group class and/or alternatively the instructor 10 can select to be a participant/instructor in one or more prescribed individualized classes. In some exemplary embodiments, the tele-instructor 10 can be required for group instruction classes, but may not be required for individualized remote sessions. Additionally, in some exemplary embodiments, a user 20 can provide a rating of the instructor 10 that was present during the prescribed group class. The instructor 10 can communicate to the use through the device and/or a separate interface such as a phone, tablet, or monitor.

In various exemplary embodiments, the movement feedback system can also include group classes in the exercise database 120 and further operate as a social platform that may be communicatively coupled with a plurality user devices 110. The user devices 110 can be in communication with a video chat platform and/or a video content distribution network that receives audio and/or video content from one or more of the user devices 110 to allow for communication between the various users in the group class or about the exercises as well as instructor 10. The networked social platform and methods may include multi-directional communication and data transfer capabilities that allow video, audio, voice, and data sharing among all users 20 through there user devices 110 and/or to instructors 10 and their interface 170. The system can further initiate and utilize virtual rooms for group exercising/therapy of patients/users that may require the same or similar treatments and/or movements. Additionally, the system can provide a rating and/or score based upon each users 20 performance. The rating can be compared to a stored database of ratings of other patients/users that have carried out similar movements and/or programs. In some exemplary embodiments the AI-monitoring system can prescribe a rehabilitation or movement program based upon the users need or injury diagnosis.

The AI movement monitoring system of the present disclosure can then identify and select one or more specific exercises or prescribed movements to generate a progress note 170 or summarized report containing the assessment of the performance of the prescribed movement based upon the biomechanical and physiological data obtained during the performance by the user 20. The AI-generated progress note 170 report can be used by the system to provide performance feedback to the user 20 and/or instructors 10. The progress note feedback report can then be utilized as a metric for future performance evaluation by the initial instructor or physical therapist and user. In some exemplary embodiments, the report can include biomechanical analysis of exercise movement parameters, including but not limited to joint range of motion, angles, muscle strength rating against gravity, displacement of body joints, the speed of joints' movement for each exercise, and the performance ratings among other biomechanical data metrics. In some exemplary embodiments, the report can include physiological analysis of each exercise performance includes but not limited to heart rate, number of steps, blood pressure, energy consumption among other physiological data metrics. The exercise database and or movements can include any suitable movements/exercises including, but not limited to gait training exercises, strength training exercises, stretches, plyometrics, active assisted, balance training, mobilization, isometric exercises, among others.

The system of the present disclosure can archive and/or transmit or provide a summary feedback report to user 20 to access prior to the next session and update the AI module 190 to aid in ensuring that the user 20 makes particular changes to their movements or to ensure that the user is properly performing the movements/programs when at their house or remote outside of a rehabilitation or training facility. The AI-driven monitoring system may then use the progress note feedback reports to assign or change the prescribed program in the exercise database 120 for the user or send it to the initial instructor/physical therapist to determine what movements may need to be directed and/or monitored during an in-person therapy/instruction session. Similarly, the reports and/or video content can be utilized to determine if movement goal parameters need to be changed, reassessed, or provide data for the instructor/physical therapist during in person sessions at the clinic to focus on various aspects of the prescribed routine as it is being performed by the user 20. In some exemplary embodiments, an instructor/physical therapist can manually review the results and adjust the program accordingly.

The system can additionally measure and compare the movement measurements in real-time of the user to the prescribed goal movement parameters for the prescribed movement. This comparison can be utilized by the system to generate the real-time feedback to the user 20 and similarly used to rate the performance of the movement by the user 20. Additionally, the system can track and compare the progress of a user's improvements in a AI-prescribed movement based upon the one or more movement parameters monitored.

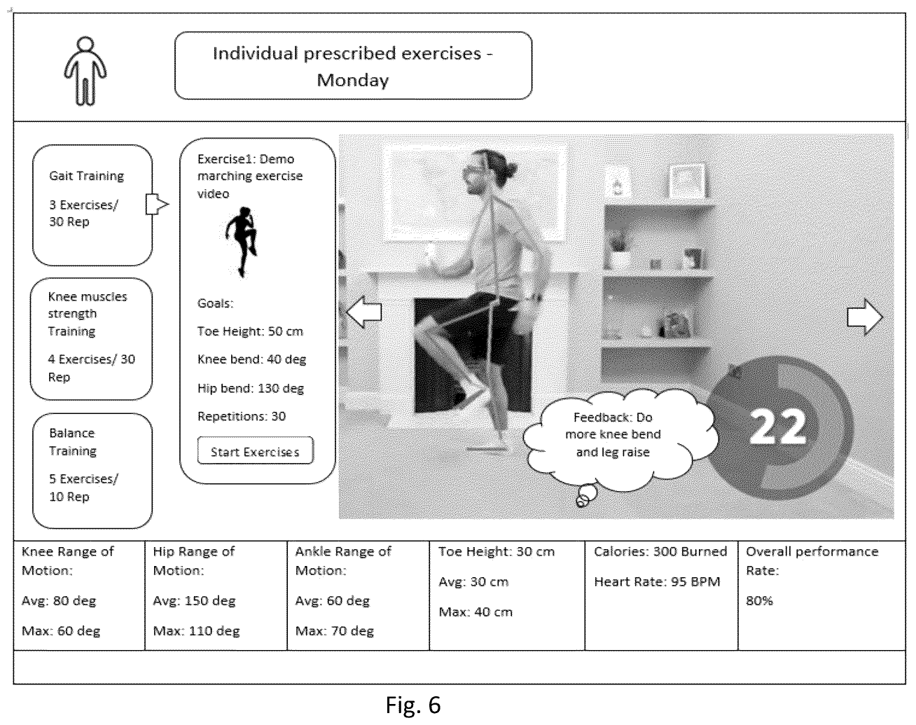

In some exemplary embodiments, the AI-movement monitoring system can be communicatively coupled to secondary physiological sensors 116, such as a patient's smart watch or health monitor to monitor and give a report for physiological analysis of each exercise performance, which includes but not limited to the heart rate, number of steps and energy consumption. The system can utilize the audio/visual data obtained during the monitoring of the user performing the prescribed program separately or in conjunction with any obtained physiological data obtained. Additionally, the system can be utilized for in person physical therapy sessions which can be recorded and utilized at home by the user 20. The system can provide side by side display of the pre-recorded in person training and guidance corresponding to the movement initiated by the system for the at home therapy session (FIG. 6). A user device 110 or a separate graphical interface can display one or more physiological readings from the secondary device configured to obtain the physiological data. The AI-movement monitoring system of the present disclosure can further communicate during and after exercises with a virtual instructor 10, such as a Tele-PT, for a higher quality of supervision and feedback if requested by the user/patient.

In some exemplary embodiments, the AI-monitoring system of the present disclosure may also initiate a question or poll to a user after performing each individual movement and/or the entire therapy program. The poll can include pain rating questions as well as other feedback questions. The system can store and utilize the subjective rating of pain after each exercise and biomechanical and physiological data from patients performed each exercise to generate one or more feedback reports/summaries progress note for review. In accordance with the prescribed programs and/or program goals set by physical therapist/instructor and the population of patients performed in each exercise, provides rating of patients' performance and pain in comparison to patients with similar complications.

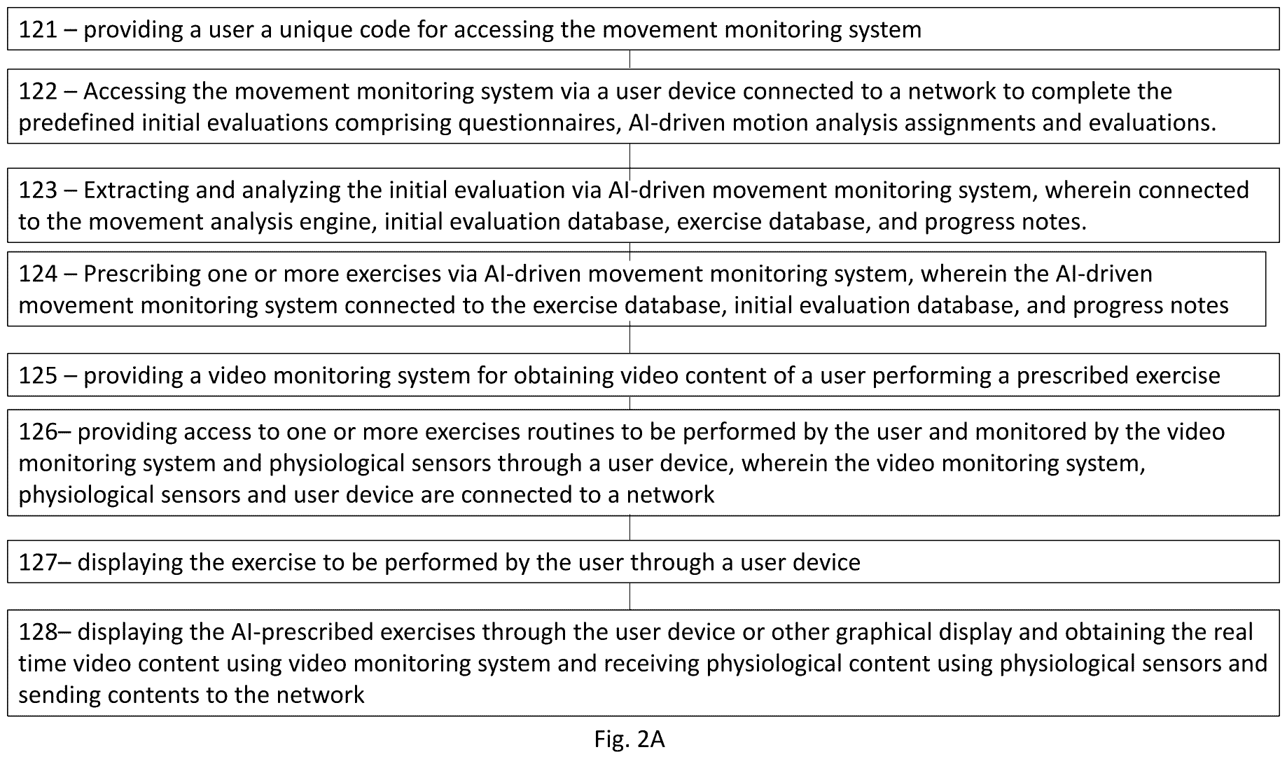

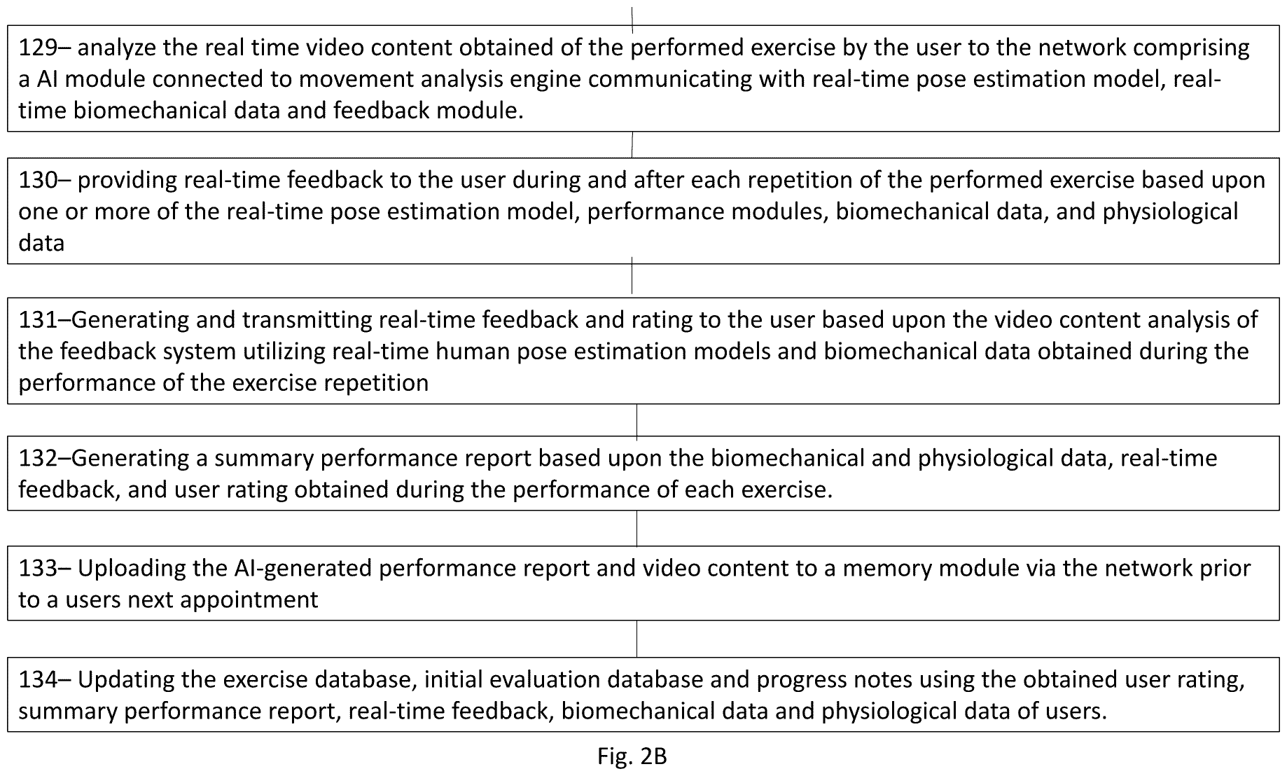

As shown in FIG. 2 the present disclosure provides a method for utilizing an AI-monitoring and prescribing of the present disclosure. The method can include first providing a user a unique code for accessing the movement feedback system (step 121). In some exemplary embodiments, the system can be accessed via network 200 or through a downloadable app on a user device 110 to complete the predefined initial evaluations comprising questionnaires, AI-driven motion analysis assignments and evaluations (step 122). An AI-guided movement analysis engine can extract and analyze initial evaluation assigned movements, wherein connected to the initial evaluation database, exercise database, and summarized feedback progress notes (step 123). Afterwards, the AI-monitoring system prescribes one or more exercises, wherein the AI-driven movement monitoring system connected to the exercise database, initial evaluation database, and summarized feedback progress notes (step 124). A video monitoring system 118 can be provided for obtaining video content of the performance of the AI-prescribed movements (step 125). In some embodiments, the user device can function as the user device, video recording devices, and a graphical display. In other embodiments, the user device, video recording device and graphical display can be separate apparatus or a combination of one or more of the various apparatuses each of which can be communicatively coupled to the other and/or to the network.

The user can then be provided access to one or more exercises routines to be performed by the user and monitored by the video monitoring system through a user device (step 126). In some exemplary embodiments, multiple users can be provided access to exercise routines. Additionally, the exercise routines can have an established time prescribed for their performance and multiple users can perform the exercises simultaneously in a virtual group class. In some exemplary embodiments, users can communicate with each other during the performance of exercise during the group class setting.

The AI monitoring system can display the exercise to be performed by the user through a graphical display (step 127). This can provide a user with a real-time guide of how to perform each exercise during the execution of the exercise movement by displaying the prescribed exercises through a user device or other graphical displays. The video content obtained during the performance of each repetition of the exercise can then be obtained by the apparatus (Step 128). The movements and real time video content can then be analyzed using a movement analysis engine/algorithm that can include a real-time pose estimation model, real-time biomechanical data, and physiological data (step 129). The data can be transmitted over the network to a server or other device for processing. Real-time feedback can then be provided to the user during and after each repetition of the performed exercise based upon the analysis of the video content by the movement feedback system. The feedback can include any suitable information, including but not limited to performance rating, audiovisual correction feedback, number of repetitions, performance rating, heart rate, calories burned, range of motion rating, among others (step 130-131). Additionally, the system can provide a performance data of each of the users in the group class during the class. The system can generate a summary performance report based upon the biomechanical and physiological data, real-time feedback, and user rating obtained during the performance of each exercise (step 132-3). Lastly, the AI apparatus can update the exercise database, initial evaluation database and summarized feedback progress notes using the obtained user rating, summary performance report, real-time feedback, biomechanical data and physiological data of users (step 134).

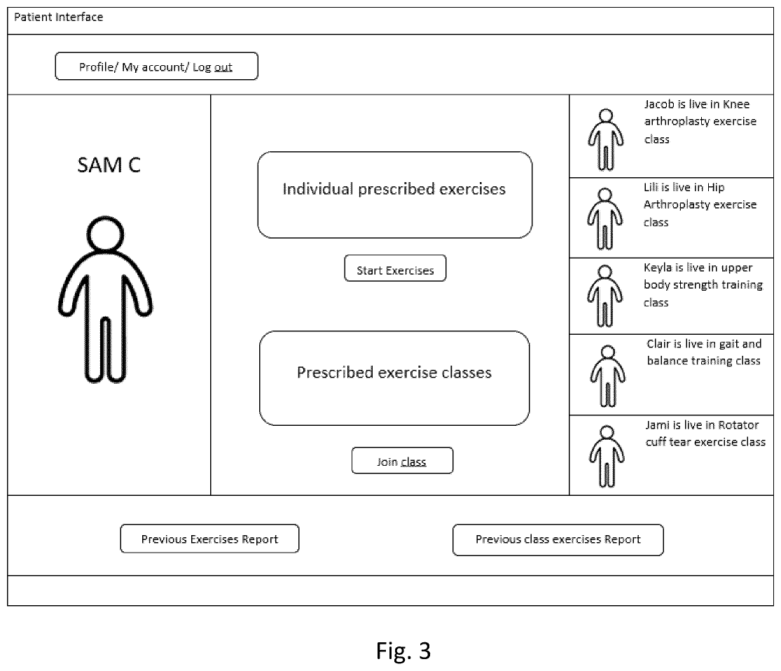

FIGS. 3-9 illustrate various graphical user interfaces that can be used by a user when utilizing the system and providing feedback to the user when utilizing the system and method of the present disclosure. The user interface may present a variety of screens to the user 20, which the user 20 can move among to manage their experience, including selecting prescribed exercise routines, group classes or sessions, historical performance data and report, social communication and comments about an exercise or class, and arranging how such information is displayed on their system. Additionally, the interface can overlay information including performance information of a user or group of users communicated during the exercising, prescribed exercise movement and target information, real-time feedback, as well as the live video of the user. The user interface can allow a user to quickly access and move through various screens to access the users desired information or program including through touchscreen access.

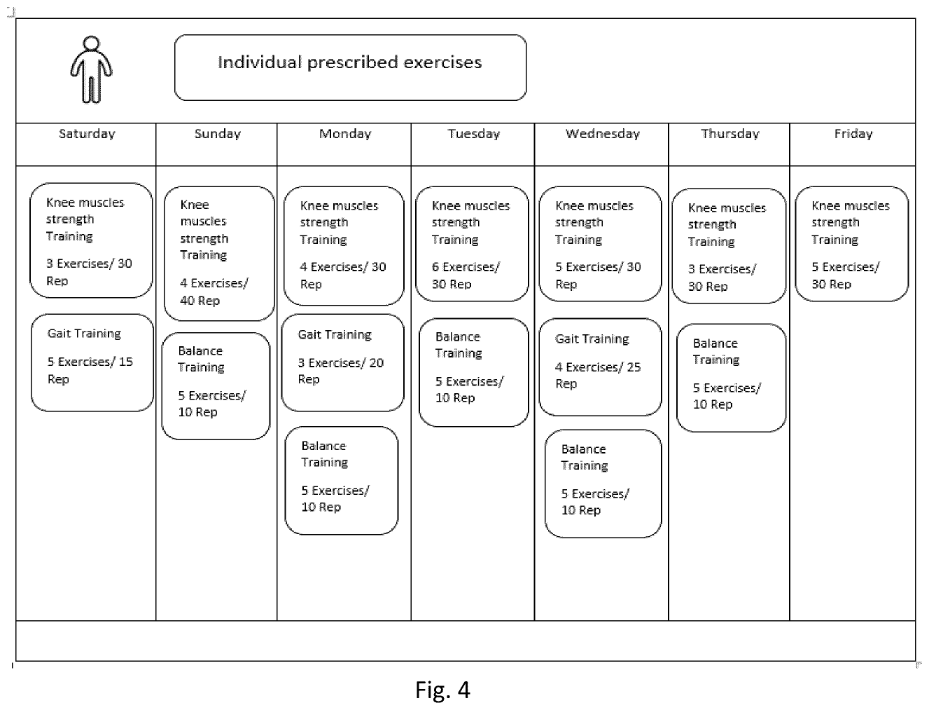

The user interface can provide a home screen that can be similar to that shown in FIG. 3 which can provide direct access and or links to the users account information and specifically prescribed individual courses and/or group courses that may be prescribed or that are available to the remote user. The home screen can also provide links to previous course performance information and/or reports and the commented feedback of our users left as a social interaction. As recited, the system can implement both individualized prescribed exercise programs/routines assigned to the user as well as live group courses that may be available, recommended, and/or prescribed to the user. The group courses can additionally include social networking information and communications between various users accessing the same group class or shared exercises. When a user selects the various class options, the user interface can be displayed in various formats including a list, calendar type (FIG. 4), or any other suitable format that provides the prescribed individual classes and/or the various available group classes and what dates those classes are either AI-prescribed or available. The user interface can similarly allow a user to sort the programs based on various parameters including but not limited to type of training, group lessons, tele-physical therapist attended, date, time, etc.

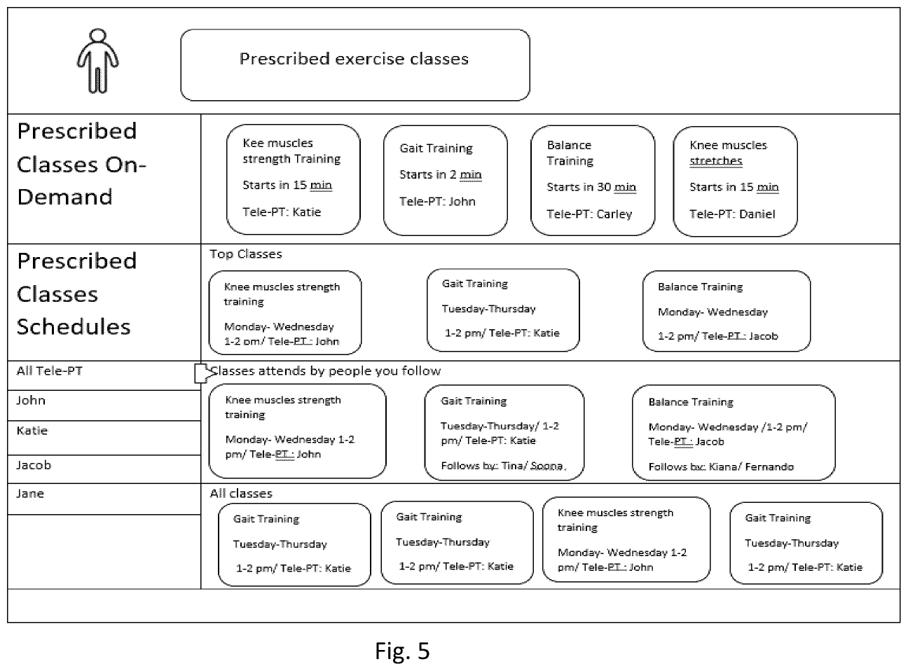

FIG. 5 provides a display provided by the interface that can provide various prescribed routines/classes to the user based upon their prescribed movements and routines as well as in relation to the social network and the classes that other similar users that they follow may be either signed up to attend or may have historically attended. These can include the assigned individual prescribed classes, assigned, prescribed, or related group classes, classes/routines that are attended by other users followed or networked by the user, and all classes available. Similarly, the interface can provide a list of classes that are available wherein a Tele-physical therapist/third-party instructor will be available upon request and attending the class/session. User lists and information may be accessed, sorted, filtered, and used in a wide range of different ways. For example, other users can be sorted, grouped and/or classified based on any characteristic including personal information such as age, gender, weight, or injury type, recover program classification, similar classes assigned/prescribed. Additionally, in some exemplary embodiments, a user can rate and/or follow the tele instructor that conducts the prescribed group courses. Similarly, the system can provide notifications to a user when a user or instructor that is being followed has signed up for a group course or signed up to be the instructor for a particular course.

As shown in FIG. 6, the system can monitor the user's movements during the performance of a prescribed movement and/or exercise. The system can provide real-time feedback based upon the video data obtained based upon the movement analysis algorithm to determine if the user is carrying out the AI-prescribed movement within a prescribed threshold for the specific user. The feedback can be auditory, visual, and/or tactile feedback to inform the user of the prescribed feedback. In some exemplary embodiments, each one or more prescribed movements can have goal parameters assigned to the movement to ensure progress and or successful completion of the movement by the user. The system can display real-time assessments/feedback which can include feedback, ratings, and related measurements for the various prescribed movements. Additionally, during the performance of the prescribed class and related assigned movements, the interface can display information regarding the prescribed movement, including but not limited to reps required, type of movement, number of exercises, target/goal movement parameters, automated generated feedback by the system, feedback provided by a tele-physical therapist/instructor, demo videos, audio, and or images illustrating proper form of the exercise and real-time completion of the prescribed program.

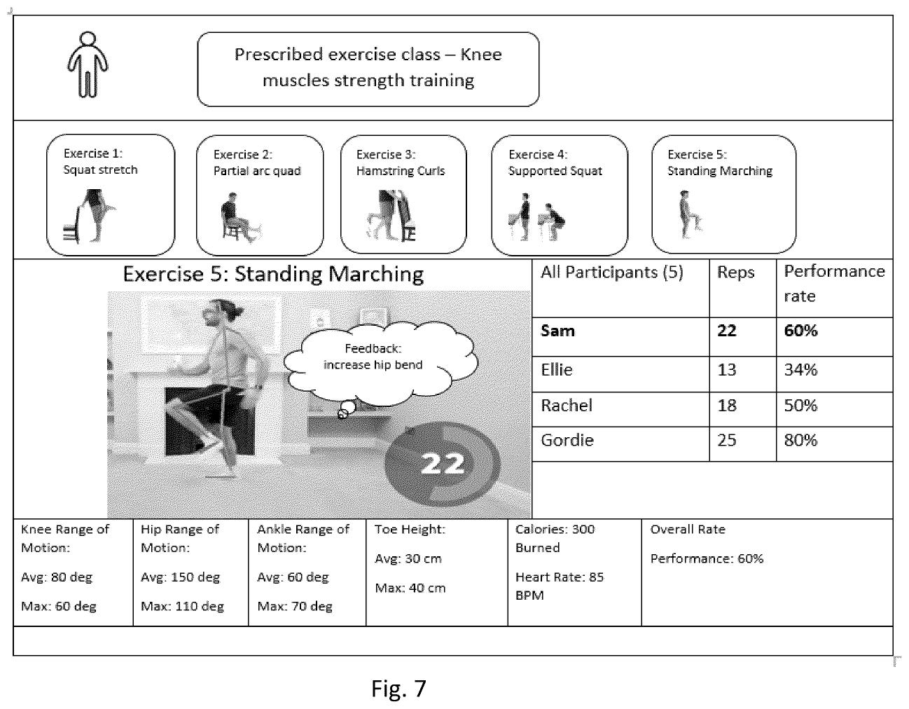

FIG. 7 illustrates an exemplary embodiment of a user interface for a group class that can include a list of the various exercises/movements included in the group class, participants of the class, reps completed by each participant, performance rating related to each participant, and the target and average movement parameters of the individual user participating in the group class and their individualized goals/target measurements as it relates to their prescribed movements. Similarly, if an exercise is not applicable or may not be part of the individuals prescribed movement or does not apply to the prescribed routine related to the individual users program assigned by a third party trainer/physical therapist the user can be instructed or it may be displayed to skip or not-participate in a particular exercise/movement.

As previously recited, the system can generate a post-performance feedback progress note report. The feedback report can provide illustrative and/or free-word assessment based upon the users performance of each of the one or more movements prescribed to perform in one session, multiple sessions, and for selection of prescribed exercises. For instance, the feedback progress note report can state that user only fully completed “x” number of repetitions of exercise 1 and out of the completed repetitions only “y” were correctly performed. Additionally, in some exemplary embodiments, a performance rating can be assigned to the user based upon the user's execution of the one or more movements comparing to other users with similar condition. The user may also provide additional feedback upon completion of the one or more exercises or at the end of the routine, such as pain associated with the one or more prescribed movements.

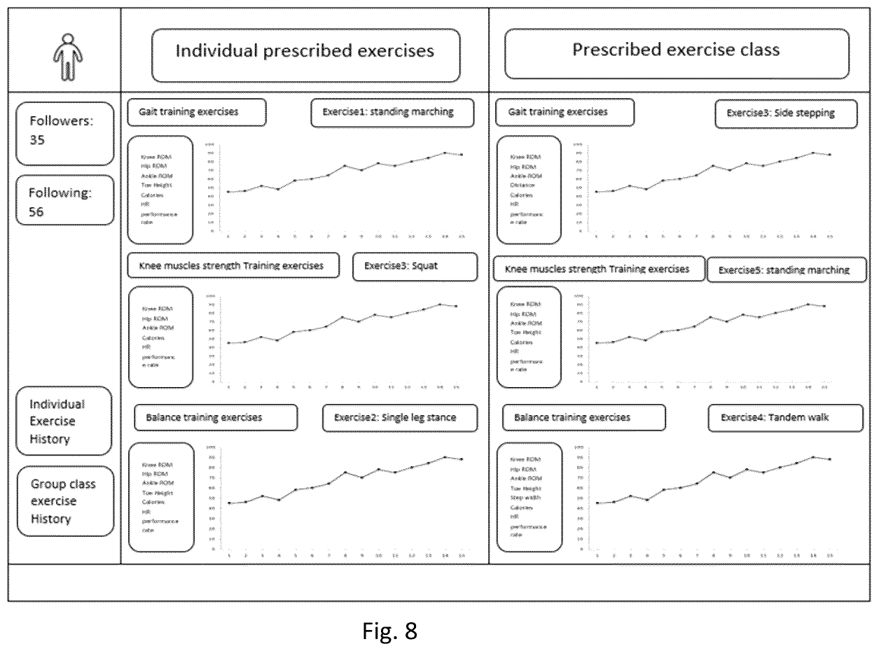

The user interface can display progress report(s) and historical performance data to a user via a display device. FIG. 8 is an exemplary embodiment of a display illustrating progress by a user over time based upon each of the prescribed exercises and the baseline or initial movement measurement and the current movement parameter or measurement related to the prescribed movement/routine. In some exemplary embodiments, the baseline measurements can be established using a contour base calibration based upon the individual users personal information including but not limited to height and other body measurements. The calibration/baseline can be set for each of the prescribed exercises/movements to be performed by the user. The progress data can be displayed in any suitable manner including graphical illustrations of the improvements in movement over a period of time.

The interface can additionally provide user information including patient ID, name, injury type, surgery type, recovery class, age, gender, and/or other statistics. Similarly, the interface can allow the user to select and assign various exercises/movements to be prescribed as well as corresponding movement parameters/thresholds/goals for the prescribed user for the prescribe movement.

In some exemplary embodiments, the system of the present disclosure can provide multimedia inputs and outputs for social interaction among users, or with physical therapist about streaming or archived prescribed programs content that can include but not limited to rehabilitation workout routines or exercise movements, socially networked audio and video chat, networked performance metrics and competition capabilities, along with a range of rating features. The system can utilize a user interface that can be communicated to a user via a display or device, including but not limited to a computer, tablet, phone, or other device. The user interface can be used to access user information, login, logout, live content for group lessons, and or individualized prescribed routines or classes assigned to the specific user. User information may be displayed in variety of formats and can include the historical and current performance information and user account information, social networking links, and ratings, etc. The user interface can also be used to access the system to update profile information and manage account settings which can include information setting standards, social network information, control settings, among other items. In the social platform, the users are able to see the following friends, followed friends, chat box, commenting and sharing video or pictures and so on. Additionally, the user interface may display a progress percentage based upon the amount of courses completed by the user in relation to the amount of courses prescribed to the user and the performance rate or relative rating to the other users with similar condition. Similarly, the progress rating may be based upon the user's mobility and/or performance of movements in relation to the target movement parameter.

While the invention has been described above in terms of specific embodiments, it is to be understood that the invention is not limited to these disclosed embodiments. Upon reading the teachings of this disclosure many modifications and other embodiments of the invention will come to mind of those skilled in the art to which this invention pertains, and which are intended to be and are covered by both this disclosure and the appended claims. It is indeed intended that the scope of the invention should be determined by proper interpretation and construction of the appended claims and their legal equivalents, as understood by those of skill in the art relying upon the disclosure in this specification and the attached drawings.

What is claimed is:

1. An artificial intelligence apparatus for prescription of exercise routines to a user; comprising:
a memory configured to store one or more inputs and user data, wherein the memory is communicatively coupled to a network;
a processor communicatively coupled to the network, configured to:
provide access to the user to complete an initial evaluation comprising a general health and performance questionnaires and initial diagnosis;
provide an access to the user to perform pre-determined AI-guided assessments, wherein a camera and one or more physiological sensors are communicatively coupled to allows user to access the assessments, to perform AI-driven assessment of joints range of motion, muscular strength and performance to generate an initial evaluation response output;
receive initial evaluation response output that comprises: a movement video and physiological data from a user for the pre-determined AI-guided assessments;
analyze the assessments movement video and physiological data with AI-module with respect to a movement database, initial evaluation database, and extracting one or more pre-determined movement parameters;
prescribe one or more exercise routines to the user, wherein an AI-prescription established with respect to movement database, movement assessments, other users progress notes, exercise database and initial evaluation database;
provide information to the user about a one or more AI-prescribed exercise routine that can be accessed via the network;
transmit to an user interface that comprises a display and a camera, wherein the display allows user to access the AI-prescribed exercise routine and the camera is configured to capture video data of the one or more movements of the user and the physiological sensor is configurated to collect performance data while participating in the exercise routine;
analyze the one or more movements with respect to movement and initial evaluation databases and one or more pre-determined movement parameters;
determine the performance of AI-prescribed movements of the user with respect to the exercise and initial evaluation databases movement parameters;
generate and provide real-time feedback to the user based upon the performance of the movement, wherein the real time feedback comprising:
an automated response from the AI-module;
real-time instruction using AI-driving module from the system comparing a measured movement parameter to a target movement parameter.
a summary of exercise performance based upon the movement of the user and analysis of the user movement or group of users,
wherein the feedback includes at least one of the following:
visual, auditory, or haptic modalities.

2. The system of claim 1, a secondary device for monitoring physiological metrics of the user.

3. The system of claim 1, wherein the modeling algorithm comprises a human pose estimation model for each movement for the movement analysis engine.

4. The system of claim 1, wherein the server can store one or more baseline measurements for each movement for the user and one or more prescribed measurements parameters for each movement.

5. The system of claim 1, wherein the summary of exercise performance includes at least one of the following:
Joint range of motion, lift height, depth, speed of movement, joint displacement, standing and walking performance scales.

6. The system of claim 1, further comprising one or more physiological sensors, wherein the physiological sensors monitors at least one of the following:
heart rate, energy exertion, number of steps, blood pressure, and blood oxygen.

7. The system of claim 1, further comprising a social network among users wherein the social network provides at least the following:
communication between users, exchanging comment, sharing video and picture, interacting with the instructor, and observing the performance and ratings of other users.

8. The system of claim 5, wherein the summary of exercise performance transmits to databases via a network.

9. The system of claim 3, wherein the modeling algorithm comprises a natural language processing for AI-generated summarized progress note report.

10. A method for an AI-generated post-performance progress note summary report, comprising:
a memory having one or more prescribed movement programs and/or classes, wherein the movement programs can include the user profile, one or more movements and one or more movement parameters, and one or more users;
the AI-generated summarized feedback generates a performance report based upon the biomechanical data of a user or multi users' performance of the performed exercise.
providing post-performance AI-generated summarized feedback report to the user upon completing the AI-prescribed exercise routine; and
communicating the post-performance feedback to the exercise and initial evaluation databases.
